(19) United States
(12) Patent Application Publication
(10) Pub. No.: US 2023/0145686 A1
(43) Pub. Date: May 11, 2023

(54) Drug Delivery Device

(71) Applicant: Sanofi, Paris (FR)

(72) Inventor: Philipp Troebner, Frankfurt am Main (DE)

(21) Appl. No.: 17/918,236

(22) PCT Filed: Apr. 23, 2021
(86) PCT No.: PCT/EP2021/060629

§ 371 Date: Oct. 11, 2022

(30) Foreign Application Priority Data
Apr. 23, 2020 (EP) 20315207.9

(51) Int. Cl. A61M 5/50; G16H 20/17; G16H 40/63; A61M 5/315; A61M 5/32

(52) U.S. Cl. CPC A61M 5/5086; A61M 5/3202; A61M 5/31551; G16H 20/17; G16H 40/63; A61M 2005/3125; A61M 2205/3569; A61M 2205/6018

(57) ABSTRACT

The present disclosure relates to a method of pairing an electronic data logging device of a drug delivery device with an external electronic device and to a respective drug delivery device. The drug delivery device comprises a housing configured to accommodate the medicament and to receive and/or mechanically engage with the electronic data logging device. The drug delivery device further comprises a first device component and a second device component. The first device component is movable relative to the second device component from a first positional state to a second positional state. The first device component comprises a first outer surface, and the second device component comprises a second outer surface. At least one of the first outer surface and the second outer surface comprises a first code portion of a pairing code for establishing a communication link between the electronic data logging device and an external electronic device.

### CROSS REFERENCE TO RELATED APPLICATIONS

The present application is the national stage entry of International Patent Application No. PCT/EP2021/060629, filed on Apr. 23, 2021, and claims priority to Application No. EP 20315207.9, filed on Apr. 23, 2020, the disclosures of which are incorporated herein by reference.

### TECHNICAL FIELD

The present disclosure relates to the field of drug delivery devices for administering of a dose of a medicament. The disclosure particularly relates to a drug delivery device operable to cooperate with an electronic data logging device, wherein the data logging device is further connectable to an external electronic device. The present disclosure particularly relates to the field of establishing or providing a communication link between a data logging device suitable for a drug delivery device and an external electronic device. The disclosure further relates to a method of pairing an electronic data logging device of a drug delivery device with an external electronic device.

### BACKGROUND

Drug delivery devices for setting and dispensing a single or multiple doses of a medicament are as such well-known in the art. Generally, such devices may have substantially a similar purpose as that of an ordinary syringe.

Drug delivery devices, such as injection or inhaling devices, e.g. pen-type injectors, have to meet a number of user-specific requirements. For instance, with patient’s suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

The medicament to be dispensed or expelled by the drug delivery device may be provided and contained in a multi-dose cartridge. Such cartridges typically comprise a vitreous barrel sealed in distal direction by means of a pierceable seal and being further sealed in proximal direction by the bung. With reusable drug delivery devices an empty cartridge is replaceable by a new one. In contrast to that, drug delivery devices of disposable type are readily equipped with such a cartridge. They are to be entirely discarded when the medicament in the cartridge has been dispensed or used-up.

With some drug delivery devices, such as pen-type injection devices a user has to set a dose of equal or variable size by rotating a dose dial in a clockwise or counter clockwise direction relative to a body or housing of the injection device for increasing or decreasing of the dose. For injecting and expelling of a dose of a liquid medicament the user has to depress a trigger or dose button in a distal direction and hence towards the body or housing of the injection device. Typically, the user uses his thumb for exerting a distally directed pressure onto the dose button, which is located at a proximal end of the dose dial and the dose dial sleeve, while holding the housing of the injection device with the remaining fingers of the same hand.

For mechanically implemented injection devices it is desirable to enable a precise, reliable and quasi-automated supervision and/or collection of injection-related data during use of the injection device. Mechanically operated injection devices may be equipped with an electronically implemented add-on device or data logging device configured to monitor user-induced operation of the injection device.

Some data logging devices may be configured for attachment to an injection device. Some data logging device may be embedded or arranged in the injection device. Other drug delivery devices may be implemented electronically, hence they comprise an integrated electronic unit configured for and operable to monitor operation of the drug delivery device over time. Here, the data logging device is provided by the integrated electronic unit.

Administration details and patient specific data can be logged and stored in such data logging devices during repeated use of the respective drug delivery device. It is generally desirable to obtain access to the respective data as stored in the data logging device, e.g. for patient supervision and/or for optimizing a long-term therapy. The data logging device may be configured to establish a communication link with an external electronic device. The communication link may be of wireless or wired type. The data collected or at least temporally stored in the data logging device might be rather sensitive and/or delicate. Misuse or unauthorized access of this data should be prevented and avoided.

In order to prevent unauthorized access to the data, establishing of a communication link between the data logging device and the external electronic device may require an authorization. Here, the data logging device and/or the external electronic device may be implemented to establish the communication link only upon confirmation or entering of a suitable key or code. Generally, such a pairing code may provide security against misuse or unauthorized access to the data stored in the data logging device, provided that the pairing code is only known to authorized personnel.

On the one hand, such a key or code should be unequivocally available to authorized personnel, e.g. to the healthcare provider or to the patient. On the other hand, such a pairing code should not be directly discernible from outside the drug delivery device to provide sufficient data security. Regulatory requirements may further specify that such pairing codes have to be visible from outside the injection device or data logging device.

It is therefore a demand to provide an improved drug delivery device configured to provide a pairing code for establishing a communication link between an electronic data logging device and an external electronic device. On the one hand, the drug delivery device should prevent unauthorized access to the pairing code. On the other hand the drug delivery device should effectively provide unobstructed and unequivocal access to the pairing code for authorized personnel.

### SUMMARY

In order to solve the above described demand there is provided an improved drug delivery device for administering of a dose of a medicament and a method of pairing an electronic data logging device of a drug delivery device with an external electronic device in accordance to the features of the independent claims. Further features of the device and the method are subject of the dependent claims.

In one aspect there is provided a drug delivery device for administering of a dose of a medicament. The drug delivery device is operable to cooperate with an electronic data logging device. The drug delivery device comprises a housing to accommodate the medicament. The housing is further configured to receive the electronic data logging device or to mechanically engage with the electronic data logging device. The housing may be configured to contain the data logging device. The data logging device may be either implemented as a separate device connectable to the housing of the drug delivery device or to a movable component of the drug delivery device. With other examples, the electronic data logging device is integrated into the drug delivery device and belongs to the drug delivery device.

The drug delivery device comprises a first device component. The drug delivery device further comprises a second device component. The first device component is movable relative to the second device component at least from a first positional state to at least a second positional state.

The first device component comprises a first outer surface. The second device component comprises a second outer surface. At least one of the first outer surface and the second outer surface comprises at least a first code portion of a pairing code. The pairing code is usable for establishing a communication link between the electronic data logging device and an external electronic device. The pairing code may be required for establishing the communication link. The pairing code may be a security code requested by at least one of the electronic data logging device and the external electronic device in order to establish the communication link. The pairing code may comprise or constitute a key required to establish the connection between the electronic data logging device and the external electronic device. Generally, the pairing code is usable or necessary for establishing a secure connection between the data logging device and the external electronic device.

The pairing code may comprise or may constitute also at least one of a bonding code or a session key. The pairing code may also serve as a password or passphrase to establish a long term authentication between the drug delivery device and the electronic data logging device.

Typically, the electronic data logging device and the external electronic device each comprise a wireless and hence mechanically contact-less communication interface. For establishing a communication link between the wireless communication interface of the electronic data logging device and the external electronic device at least one of the wireless communication interfaces is configured to execute a pairing routine or pairing procedure, e.g. based on a well-defined wireless communication standard.

Generally, the communication link and the way of communication between the data logging device and the drug delivery device is not limited to a particular wireless communication technology wireless communication standard. The present disclosure is generally applicable to all available types of mechanically contactless communication links that require authentication for and/or during establishing a secure connection between the communicating interfaces, e.g. of the data logging device and of the drug delivery device, respectively.

With some examples, the communication link is an electromagnetic communication link, that may provide or support contactless charging, radiofrequency based communication, such as WiFi, Bluetooth, RFID, NFC, GSM, UMTS, LTE or others. With further examples the communication link is based on optical transmission in the infrared or visible spectral range. With further examples the communication link is based on acoustic signals.

Execution of the pairing routine comprises an authentication step. Here, at least one of the external electronic device and the electronic data logging device is operable to request a pairing code associated with the other one of the external electronic device and the electronic data logging device. With some embodiments a pairing code, typically associated with the electronic data logging device, may be communicated or transmitted to the external electronic device. Before the respective communication link can be established, a user may be prompted to confirm the pairing code, either by entering the pairing code into an input interface of at least one of the electronic data logging device and the external electronic device. With other embodiments, at least one of the electronic data logging device and the external electronic device may be operable to visually or audibly present the pairing code, which has to be confirmed and/or approved by a user of the respective device before the communication link will be established.

With some examples the pairing code provided on the drug delivery device characterizes the drug delivery device and/or the medicament intended for the drug delivery device or arranged inside the drug delivery device. Hence, drug delivery devices of different type may distinguish from each other by different pairing codes. Identical drug delivery devices, i.e. drug delivery devices equipped with the same medicament may be provided with a common or with the same pairing code.

The pairing code may contain unique information or a unique identification of the medicament located inside the drug delivery device. This particularly applies, where the drug delivery device is a prefilled drug delivery device, e.g. a prefilled injection device, which is to equipped with only one type of a medicament.

By arranging or integrating the data logging device onto or into the housing of the drug delivery device the data logging device may keep track of a configuration of the drug delivery device. Upon pairing of the data logging device with the external electronic device and by e.g. entering or confirming the pairing code provided on the drug delivery device into or with the external electronic device, the external electronic device is inherently provided with the unique information or unique identification of the medicament located inside the drug delivery device.

Moreover, and alternatively, e.g. when the electronic data logging device is fixedly and non-detachable arranged inside or onto the drug delivery device the electronic data logging device may be preprogrammed and provided with sufficient information allowing to identify the type of the medicament and/or the type of the drug delivery device to which the electronic data logging device is actually attached to. Moreover, upon or during integration or assembly of the electronic data logging device into or with the drug delivery device, the pairing code can be provided on the first and/or second device components as well as in an electronic memory of the electronic data logging device. Also, there can be provided a unique assignment between the pairing code provided on the drug delivery device itself and irrespective pairing code stored in a memory of the electronic data logging device.

With some examples, the pairing code comprises at least 3 digits, at least 4 digits, at least 5 digits, at least 6 digits or any larger number of digits. Here, a digit may comprise a number, a letter or any other visually identifiable sign.

The pairing code is visually identifiable from outside the drug delivery device only when the first device component is in the second positional state relative to the second device component. Hence, when and as long as the first device component is in the second positional state the pairing code is discernible from outside the drug delivery device. In all other positional states, e.g. in the first positional state or in further positional states of the first device component relative to the second device component the pairing code is non-discernible from outside the drug delivery device.

Here, the pairing code or at least a portion thereof may be either concealed or covered by at least one of the first or second device component. With some examples, the pairing code may be visible from outside the drug delivery device even when the first device component is outside or offset from the second positional state. Here, the pairing code may be provided with a supplemental code, such as a concealing code portion indistinguishable from the pairing code. Even though the code may be visible it cannot be unequivocally identified visually as long as the first device component is outside the second positional state relative to the second device component.

The drug delivery device may be implemented as an inhaler, as an injection device or as an infusion device. The drug delivery device may be implemented as a pen-type injector. The electronic data logging device may be either integrated into the drug delivery device or it may be provided as a separate data logging device or add-on device configured for releasable or non-releasable attachment to the drug delivery device. Typically, the data logging device comprises at least one sensing arrangement configured to detect and/or to quantitatively determine setting and/or dispensing of a dose of the medicament. Typically, the data logging device comprises an integrated circuit, e.g. provided on a printed circuit board (PCB). The data logging device typically comprises a processor and a memory. The data logging device further comprises a communication interface. The communication interface may comprise a wireless transceiver in order to establish a wireless communication link to the external electronic device.

The external electronic device may be implemented as a personal digital device, such as a smart watch, an activity watch, a mobile phone, a tablet computer or a personal computer. The data logging device and the external electronic device may be implemented to establish a wireless communication link in accordance to a standardized wireless communication protocols, e.g. based on radio frequency communication, such as RFID, and NFC or Bluetooth.

A pairing routine of the electronic data logging device and the external electronic device is particularly implemented to request or to confirm a pairing code. The pairing code as provided by the drug delivery device may either have to be entered into at least one of the data logging device and the external electronic device. With some examples, the pairing code may be visually displayed to the user of the drug delivery device, e.g. by at least one of the data logging device and the external electronic device. The user may then be prompted to confirm, that the illustrated pairing code is identical with the pairing code provided on the drug delivery device. In this way, misuse of data stored in the electronic data logging device can be effectively prevented.

The pairing code which might be necessary to establish the communication link is only visually identifiable or discernible from outside the drug delivery device when the first device component is in the second positional state relative to the second device component. With all other available positional states of the first device component relative to the second device component the pairing code is either concealed or it cannot be distinguished from other code information provided on the outside of the first device component and/or the second device component.

In this way, the improvement of the drug delivery device provides two alternative approaches. With one approach, the pairing code provided on at least one of the first device component and the second device component is at least partially covered by the other one of the first and the second device components when and as long as the first device component is outside the second positional state or does not overlap with the second positional state relative to the second device component. With this approach, the first and the second device components are typically displaceable relative to each other in an interleaved manner.

Hence, that component of the first and second device components which is provided with at least the first code portion is at least partially received inside the other one of the first and second device components. Hence, at least a fraction of the first code portion provided on an outer surface of at least one of the first and second device component is concealed by the other one of the first and second device components. Here, first and second device component mutually overlap. They may be arranged in an interleave or at least partially nested manner. In the first positional state, the degree of overlapping is comparatively large, thereby covering or concealing at least a fraction of the first code portion. When in the second positional state, the degree of mutual overlap is reduced so as to reveal the first code portion or the entirety of the pairing code.

According to the second alternative approach, the pairing code is permanently visible on the outside of the first and second device component. Here, the pairing code is provided with a supplemental code, hence with a concealing code portion that is indistinguishable from the pairing code. As long as both, the pairing code and the supplemental concealing code portion are both visible on the outside of the drug delivery device the pairing code cannot be visually identified because the respective user does not know the total length and the position of the relevant pairing code in or from the indistinguishable combination of the pairing code and the concealing code.

Here, the pairing code made can be hidden among supplemental concealing code portions. It is only upon moving the first device component into the second positional state relative to the second device component, that the supplemental and/or concealing code portion(s) are concealed or covered so that the residual pairing code becomes discernible from outside the drug delivery device.

In the present context a positional state between first and second device component includes a relative position as well as a relative orientation between first and second device components. In the first and second positional states, at least one of the position and the orientation of the first device component differs from the respective position or orientation of the second device component.

First and second device components may be mechanically engaged either directly or indirectly. Furthermore, first and second device components may be one of threadedly engaged or slidably engaged. The first and the second device component may be rotationally engaged or may be rotationally locked.

A movement of the first device component from the first positional state towards and into the second positional state may comprise at least one of a rotational motion, a sliding motion or a helical motion, hence a combined rotating and sliding motion. The first and the second device components may be permanently connected, e.g. by a threaded engagement or by a slotted link.

The first positional state of first and second device components may coincide with an initial state of the respective component, e.g. in which the drug delivery device is handed out to the patient or customer. The second positional state of first and second device components may coincide with a particular device configuration. With some examples preparing of the drug delivery device for administering a dose of the medicament requires a movement of the first device component relative to the second device component. With some examples, setting of a dose of the medicament requires moving of the first device component relative to the second device component; or vice versa. With some examples expelling or dispensing of the dose of the medicament requires movement of the first device component relative to the second device component.

The second positional state of the first device component relative to the second device component is a characteristic relative position and/or relative orientation of the first device component relative to the second device component. The user of the drug delivery device is well aware of this characteristic portion and has only little difficulties to move the first device component relative to the second device component to arrive at the second positional state.

Typically, the second positional state, in which the pairing code is visually identifiable or in which the pairing code is discernible from outside the drug delivery device does not coincide with an initial device configuration. Making the pairing code visually identifiable from outside the drug delivery device always requires a well-defined user induced movement of the first device component relative to the current device component.

Generally, it is only relevant, that the first device component is moved relative to the second device component. Whether or not the first device component is actually subject to a movement relative to the environment is quite irrelevant. For arriving at the second positional state the first device component may be fixed, e.g. by a hand of a user while the second device component is moved relative to the first device component.

According to a further example the first code portion or the pairing code is provided on the first outer surface. At least a part of the first code portion or at least a part of the pairing code is concealed by the second device component when the first device component is outside the second positional state. In this way and as long as the first device component is outside or nonoverlapping with the second positional state the entirety of the pairing code is not yet visually identifiable from outside the drug delivery device. Here, the respective portion of the first outer surface provided with the first code portion or provided with the entirety of the pairing code is covered by the second device component. Typically, the second device component or at least a portion thereof spatially overlapping with the first device component is opaque.

In this way the first code portion or the entirety of the pairing code is revealed when the first device component arrives in the second positional state relative to the second device component. As long as the first device component and hence the first code portion or the entirety of the pairing code is in the first positional state, at least a fraction of the first code portion or a respective fraction of the entirety of the pairing code is non-discernible from outside the drug delivery device. In this positional state and as long as the first device component is outside the second positional state the pairing code in its entirety cannot be visually identified from outside the drug delivery device. A communicative pairing between the electronic data logging device, e.g. attached to or integrated into the drug delivery device and the external electronic device is not possible and data stored in the electronic data logging device is protected against misuse.

According to another example the first outer surface is provided with one of the first code portion and the pairing code. The first outer surface is further provided with a first concealing code portion. The concealing code portion is indistinguishable from the first code portion or from the pairing code. An indistinguishable composition of at least a portion of the concealing code portion and at least one of the first code portion and the pairing code is discernible from outside the drug delivery device when the first device component is outside the second positional state, e.g. when the first device component is in a third positional state relative to the second device component.

The concealing code portion may be of the same type of code compared to the pairing code. With some examples, the pairing code may comprise a string of numbers or characters. The concealing code portion may comprise a comparable string of numbers or characters. The font, size and style of the strings of the pairing code and the concealing code potion may be identical or equivalent, hence indistinguishable. The concealing code portion may be directly aligned with the first code portion and/or with the pairing code.

The first code portion or the pairing code may be embedded or framed by the first concealing code portion. The user may not be aware about the length of the string of the composition of the first concealing code portion and at least one of the first code portion and the pairing code. The pairing code is hence blended or intermixed with the concealing code portion and becomes visually indistinguishable as long as both, the pairing code or the first code portion thereof is provided together with the first concealing code portion on the first outer surface.

With some examples, the first code portion or the entirety of the pairing code may be totally invisible and may be hence entirely concealed by the second device component when the first device component is in the first positional state. As the first device component is moved from the first positional state towards the second positional state, the first code portion or the entirety of the pairing code becomes successively visible.

When reaching the second positional state, the relevant first code portion or the entirety of the pairing code may be revealed from the second device component. As the second device component is moved further in the same direction towards a third positional state the concealing code portion provided on the first outer surface in addition to the first code portion and/or the entirety of the pairing code becomes visible and is revealed.

Since the first concealing code portion is generally indistinguishable from the first code portion or from the pairing code, the combination of the first concealing code portion with the first code portion and/or with the pairing code is effectively unusable for the user because the relevant pairing code or the relevant code portion thereof cannot be an identified in the indistinguishable code composition as provided on the first outer surface of the first device component.

This provides a further safety feature against misuse or unauthorized access to the data stored in the electronic data logging device with. Visually identifying the entirety of the pairing code requires knowledge about the rather specific and well-defined second positional state of the first device component relative to the second device component.

According to another example the first concealing code portion is concealed by the second device component when the first device component is in the second positional state. Here, the first concealing code portion may be masked or shielded by the second device component. Consequently and when in the second positional state the relevant portion, hence the first code portion or the entirety of the pairing code becomes distinguishable from or in the composition of the first concealing code portion with at least one of the first code portion and the pairing code.

According to another example the pairing code comprises the first code portion and a second code portion. The first code portion is provided on the first outer surface. The second code portion is provided on the second outer surface. Here, the entirety of the pairing code is split among the first and the second device components. The first code portion is provided on the first outer surface of the first device component and the second code portion is provided on the second outer surface of the second device component. The entirety of the pairing code is a combination of the first code portion and the second code portion. Hence, for visually identifying the entirety of the pairing code, the first code portion and the second code portion have to recombine and/or have to complement each other.

With this example, both, the first code portion and the second code portion may be permanently visible from outside the drug delivery device. For recombining the pairing code from the first code portion at the second code portion it may be necessary to arrange the first code portion and the second code portion in a well-defined or predefined manner. With some examples, the first code portion and the second code portion have to be aligned in a particular configuration only known to the user of the drug delivery device.

With some examples only one of the first and second code portions may be permanently visible on one of the first and second outer surfaces, respectively. Moving of the first device component relative to the second device component may lead to a modification of a mutual degree of spatial and non-translucent overlap of first and second outer surfaces.

Hence, in the first positional state only the second code portion may be visible from outside the drug delivery device whereas the first code portion or a fraction thereof remains revealed by the second device component. Moving of the first device component relative to the second device component into the second positional state comes along with a movement of the first code portion relative to the second code portion. Here, the first code portion may be revealed from the second device component.

In addition, the first code portion may complement the second code portion to constitute or to recombine the pairing code when the first device component is in the second positional state relative to the second device component.

The first code portion and the second code portion mutually recombine and constitute the pairing code when the first device component is in the second positional state.

With other configurations and when the first device component is outside the second positional state relative to the second device component the combination of the first code portion visible from outside the drug delivery device and the second code portion as visible from outside the drug delivery device may also mutually align or recombine but may be inoperable and may not provide the pairing code. In such positional states of the first device component and the second device component that distinguish or differ from the second positional date a recombination of first and second code portions distinguishes from the pairing code and does therefore not represent or reconstitute the pairing code required for setting up or to establish the communication link between the electronic data logging device and the external electronic device.

According to another example at least one of the pairing code and the first code portion extends along a first direction on at least one of the first and second device components, hence on at least one of the first outer surface and the second outer surface. Here, the first device component is movable along the first direction relative to the second device component. This is of particular use with e.g. the pairing code or the first code portion is provided on the first outer surface and wherein the first outer surface of the first device component is at least partially covered by the second device component, e.g. at least when the first device component is in the first positional date. As the first device component is moved towards the second positional state, more and more of the first outer surface made be revealed from the second device component.

With some examples at least a portion of the first device component may be located inside the second device component when in the first positional state. As the first device component is moved towards the second positional state, the first outer surface may continuously protrude from an end of the second device component or the respective first outer surface may extend into an aperture of the second device component, such that more and more of the first code portion or of the entirety of the pairing code becomes discernible and visible either by protruding from an end of the second device component or by appearing in the aperture of the second device component.

As the first device component is moved along the first direction relative to the second device component while being subject to a movement from the first positional state towards the second positional state a constantly increasing portion of the first code portion and the pairing code becomes discernible.

According to a further example the second outer surface of the second device component is further provided with a second concealing code portion. The second concealing code portion is indistinguishable from the second code portion provided on the second outer surface. The first code portion aligns with the second code portion when the first device component is in the second positional state. As long as the first device component is outside the second positional state, the first code portion may be offset from the second code portion. Here, the first code portion may align with a portion or with a fraction of the second concealing code. A combination and alignment of the second concealing code or a portion thereof with the first code portion may not constitute or recombine the pairing code.

According to a further example the second concealing code extends along a second direction on the second outer surface. Typically, the second direction may distinguish from the first direction, along which the first code portion extends. With some examples, the second concealing code may extend along a tangential direction of a tubular shaped and/or elongated second device component. The first code portion may extend along a first direction, which may coincide with a longitudinal axis of the elongated, e.g. tubular shaped first or second device component.

When the first code portion extends along a first direction and when the second code portion and/or the second concealing code extends along a second direction, wherein first and second directions extend at a predefined, non-zero angle relative to each other, recombining and constituting the pairing code may require that the first device component is moved from the first positional states towards the second positional state along the second direction. A movement of the first device component relative to the second device component along the first direction may be then only optional.

The first and second directions may extend perpendicular to each other. The pairing code may be recombined by a straight-line intersecting at least one of the first code portion and the second concealing code portion and a fraction of the other one of the first code portion and the second concealing code portion.

According to another example the first code portion or a fraction thereof aligns with the second concealing code portion when the first device component is outside the second positional state. The alignment of the first code portion with a fraction of the second concealing code portion is ineffective to recombine or to constitute the pairing code. When and as long as the first device component is outside of the second positional state the first code portion may exclusively align with the second concealing code portion or with a fraction thereof, thus effectively impeding to visually identify the pairing code in an overlap of the first code portion and the second concealing code portion.

According to a further example at least one of the second concealing code portion and the second code portion extends along a second direction. The first device component is movable along the second direction relative to the second device component. With some examples, the first direction extends along a symmetry axis or along a longitudinal axis of the housing of the drug delivery device. The second direction may extend in tangential or circumferential direction with regards to such an elongated, e.g. tubular shaped housing. The first and the second directions may extend at a predefined non-zero angle with respect to each other. The first and second directions may extend perpendicular to each other.

According to another example the drug delivery device comprises a first marker and a second marker. The first marker is provided by or is connected to the first device component. The second marker is provided by or is connected to the second device component. The first marker may be provided on the first outer surface. The second marker may be provided on the second outer surface. The first marker is subject to a movement relative to the second marker when the first device component moved relative to the second device component.

The first marker and the second marker visually align, visually overlap or mechanically engage when the first device component reaches the second positional state relative to the second device component. By means of the first marker and the second marker, the second positional state of the first device component and the second device component can be identified. At least one of the first and second markers is permanently visible to a user of the drug delivery device. The other one of the first and the second marker may be temporally concealed by the other one of the first and second respective device component.

Upon movement of the first device component relative to the second device component and upon reaching of the second positional state the first and second markers mutually align, visually overlap or mechanically engage, thereby indicating to the user, that the second positional state has been reached and that the pairing code is now visually identifiable from outside the drug delivery device.

With some examples, the first marker comprises an identification feature on the first surface of the first device component. The first marker may comprise a particular character or a visual sign, such as a letter or a number. The second marker may comprise a corresponding visual sign, character or number, e.g. on the second outer surface. With some examples, the second marker may comprise an aperture in the second device component. The second marker may also comprise a pointer. When for instance the second device component is the housing of the drug delivery device or when the second device component forms part of the housing of the drug delivery device it may comprise an aperture, through which at least a portion of the first device component, typically at least a portion of the first outer surface provided with the first marker can be visually identified or detected.

With some examples the first and the second marker comprise mutually corresponding and/or complementary shaped engaging features, such as a protrusion and a corresponding recess. In this way the first and the second marker may mechanically engage when the first and second device components reach the second positional state.

With some examples, the drug delivery device comprises a number sleeve with a sequence of numbers printed thereon. The sequence of numbers is indicative of a size of a dose actually set. The first marker may correspond to a particular predefined dose size, e.g. it may be represented by a dose size of a predefined number, e.g. of a dose size of 25 units. Here, the number sleeve may represent the first device component. The second device component may be represented by the housing of the drug delivery device, e.g. by a body of the housing or by a cartridge holder.

When implemented as the body of the housing of the drug delivery device the second device component may comprise an aperture or a window, through which at least a portion of the first device component located underneath is visible. With the first marker, e.g. in form of a particular and predefined dose size shows up in the aperture or in the dose window of the second device component, hence in the dosage window of the housing or body of the drug delivery device, this might be a clear indication to a user of the drug delivery device that the first device component has reached the predefined second positional state, in which the pairing code is discernible from outside the drug delivery device.

According to further examples the first device component is one of the dose dial, a dial extension, a number sleeve and a protective cap of a pen-type injection device. A dose dial is typically subject to a rotating movement during and/or for setting of a dose of variable size. During setting of a dose, the dose dial is typically subject to a rotation relative to the housing of the drug delivery device. The dose dial may be also subject to a combined longitudinal sliding movement relative to the housing, e.g. serving as the second device component according to the present terminology. Moreover, the dose dial may be subject to a combined rotational and longitudinal motion relative to the housing or body of the drug delivery device. Hence, the dose dial may be subject to a helical movement relative to the housing or body as a dose is set.

The same may apply to a dial extension. The dial extension may be subject to a longitudinal sliding motion during setting of a dose and/or during dispensing or expelling of the dose of the medicament. The dial extension may be subject to a combined longitudinal sliding movement and a rotational movement. A dial extension or components thereof may be subject to a helical movement relative to the housing of the drug delivery device, e.g. during and/or for setting or expelling of the dose of the medicament. The number sleeve may form part of a dial extension.

With some examples the number sleeve remains inside the housing of the drug delivery device. The number sleeve is rotationally supported in or on the housing of the drug delivery device. With some examples the number sleeve is locked in longitudinal direction relative to the housing. With further examples of the number sleeve is threadedly engaged with the housing. It may then follow a helical path relative to the housing when subject to a movement relative to the housing.

With other examples the protective cap configured to cover a dispensing or expelling end of the drug delivery device may be implemented as the first device component in accordance to the above terminology. The protective cap may be rotationally supported on the distal end of the housing of the drug delivery device. It may be freely oriented in numerous rotational states relative to the housing. The protective cap may be detachably locked to the housing with regard to the longitudinal direction. The protective cap may be clipped or snap fitted to the housing.

With other examples the second device component is one of a housing, a body of the housing and a cartridge holder of a pen-type injection device.

With some examples, the second device component may comprise a gauge element slidably supported in the housing of the drug delivery device and rotationally locked to the tubular shaped housing of the drug delivery device. The gauge element may be threadedly engaged, e.g. with a number sleeve. The gauge element may comprise a window or an aperture, through which a portion of the number sleeve is discernible or visible. With the implementation of a gauge element the number sleeve may be locked in longitudinal direction to the housing of the drug delivery device. It may be free to rotate relative to the housing. The gauge element may also belong to or may form part of a dial extension.

The gauge element acting as the second device component itself may be slidably supported in a longitudinal aperture of the housing. Here, the housing may act as the first device component. The gauge element may comprise a recess or a gauge aperture, through which a selected portion of the number sleeve located underneath is revealed. When threadedly engaged with the number sleeve, a rotation of the number sleeve leads to a longitudinal displacement of the gauge element relative to the aperture of the housing.

An outside surface of the gauge element may be provided with at least the first code portion of the pairing code. By setting of a dose of prescribed or predefined size the gauge element, i.e. the second device component is moved relative to the housing, i.e. relative to the first device component. In this way, the first device component may reach the second positional state relative to the second device component; and vice versa. In this positional state, at least the first code portion or the entire code as provided on the outside surface of the gauge element may become discernible in the longitudinal aperture of the housing.

During dialing or setting of a dose, the number sleeve may be subject to a rotational motion relative to the housing. Due to the threaded engagement with the number sleeve and the rotational interlock with the housing, the gauge element may slide along the rotating number sleeve, thus visually illustrating a sequence of consecutive numbers or dose size indications showing up in window or aperture of the gauge element.

A body of the housing is typically a proximal portion of the housing. The body may be configured to house or to accommodate a drive mechanism of the drug delivery device. The body may be either non-releasably or detachably connected to a cartridge holder of a pen-type injection device. The cartridge holder may be particularly adapted to accommodate a cartridge filled with a medicament. With disposable devices, a cartridge filled with a medicament is readily arranged inside the housing, typically inside the cartridge holder. The cartridge holder may be non-releasably or non-detachably connected to the body of the housing of the drug delivery device.

The above described concept of providing first and second device components of a drug delivery device movable relative to each other can be mapped and transferred to numerous components of a pen-type injection device. For instance, a first code portion may be provided at or near a proximal end of the protective cap and a consecutive second code portion may be provided at or near a distal end of the body of the housing of such a pen-type injector. The protective cap may be free to rotate with regard to the longitudinal axis relative to the body. Only in one or some further generally available positional states of the protective cap and the body the respective first and second code portions mutually align thus making the pairing code visually identifiable from outside the drug delivery device. With other configurations, the protective cap may be suitable to cover at least a portion of the pairing code when assembled or attached to the body. Detaching of the protective cap may then reveal the pairing code.

According to another example of the drug delivery device a cartridge filled with a medicament is arranged inside the housing. In this way, the drug delivery device is a drug device combination. It may be implemented as a disposable device with the cartridge readily assembled therein. It may be also configured and implemented as a reusable device. Here, the cartridge holder is detachably connected to the proximal body of the drug delivery or injection device thus allowing to replace an empty cartridge by a new, hence a filled one.

According to a further example the electronic data logging device is integrated in the drug delivery device. The data logging device may be integrated and/or assembled inside the drug delivery device, inside the housing of the drug delivery device or inside a dial extension of the drug delivery device. With other examples, the electronic data logging device is provided as a separate data logging device. It may comprise at least one fastening feature to engage with a correspondingly or complementary shaped fastening feature at a predefined position of the drug delivery device.

When implemented as a separate electronic data logging device, the data logging device may be attachable or may be readily attached e.g. to a proximal end of the drug delivery device. With some examples, the data logging device is attached to a dial extension and/or to at least one of a dose dial and a trigger of a pen-type injection device.

According to another aspect the present disclosure relates to a method of pairing an electronic data logging device of a drug delivery device with an external electronic device. The drug delivery device comprises a first device component movable relative to a second device component. The first device component comprises a first outer surface. The second device component comprises a second outer surface. At least one of the first outer surface and the second outer surface comprises at least a first code portion of a pairing code. The pairing code being visually identifiable from outside the drug delivery device only when the first device component is in a second positional state relative to the second device component.

The method comprises the step of setting at least one of the external electronic device and the data logging device into a pairing mode. Before, during or thereafter the first device component is moved relative to the second device component from a first positional state, in which the pairing code is indiscernible, into the second positional state, in which the pairing code is visually identifiable. Thereafter and when the first device component has reached the second positional state relative to the second device component the pairing code as it is now visually identifiable from outside the drug delivery device is actually visually identified or captured, e.g. by a person.

The identified pairing code is then used for establishing and/or for setting up a communication link between the external electronic device and the data logging device. Depending on the specific implementation of the pairing routine the user may be either prompted to enter the pairing code, e.g. into the external electronic device or into the data logging device. With other examples, the pairing code may be presented by at least one of the data logging device and the external electronic device, either visually or audibly. The user may be then prompted to confirm the pairing code. In case that a pairing code proposed by any one of the data logging device and the external electronic device in the course of pairing these two devices should not match with the pairing code visually identifiable from outside the drug delivery device the user should abort and cancel the pairing routine in order to prevent misuse or unauthorized use of the data stored in the data logging device.

It should be noted further, that the present method of pairing an electronic data logging device with an external electronic device with the help of the drug delivery device is universally applicable and executable by any type of a drug delivery device as described above. Therefore, any features, benefits and effects as described above in connection with the drug delivery device equally apply to the method of pairing; and vice versa.

According to a further aspect, the present disclosure relates to a medicament injection system. The injection system comprises a drug delivery device or an injection device as described above. The injection system further comprises a data logging device, either integrated into the drug delivery device or attachable to the drug delivery device. Here, the data logging device is configured and implemented to establish or to set up a communication link, e.g. a wireless communication link, to an external electronic device, such as a smart watch, a mobile phone, a tablet computer or a personal computer. Insofar, all features, effects and benefits as described above equally apply to the medicament injection system.

According to another aspect, the medicament injection system further comprises the external electronic device configured to establish and/or to set up a communication link, e.g. a wireless communication link, to the data logging device.

The injection system further comprises an external electronic device configured to establish a communication link to the data logging device.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about -4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (antidiabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N- tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl- ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide / HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of the injection device and the method of pairing a data logging device with an external electronic device will be described in greater detail by making reference to the drawings, in which:

FIG. 1 schematically illustrates an example of a drug delivery device implemented as an injection device,

FIG. 2 schematically illustrates numerous components of the drug delivery device of FIG. 1,

FIG. 3 is a schematic block diagram of an example of an electronic data logging device,

FIG. 4 schematically illustrates an electronic data logging device integrated into a drug delivery device,

FIG. 5 schematically illustrates a portion of the drug delivery device with first and second device components carrying a pairing code portion for setting of a communication link between the data logging device and the external electronic device, wherein first and second device components are in a first positional state,

FIG. 6 shows the example of FIG. 5, wherein first and second device components are in a second positional state,

FIG. 7 schematically illustrates another example of a drug delivery device provided with a pairing code, wherein first and second device components are in a first positional state,

FIG. 8 shows the example of FIG. 7, wherein the first and second device components are in a second positional state,

FIG. 9 shows first and second device components of FIGS. 7 and 8 in a third positional state, and

FIG. 10 shows a flowchart of executing a method of pairing an electronic data logging device with an external electronic device by using a pairing code as provided by the drug delivery device in accordance to any of the FIGS. 1-9.

### DETAILED DESCRIPTION

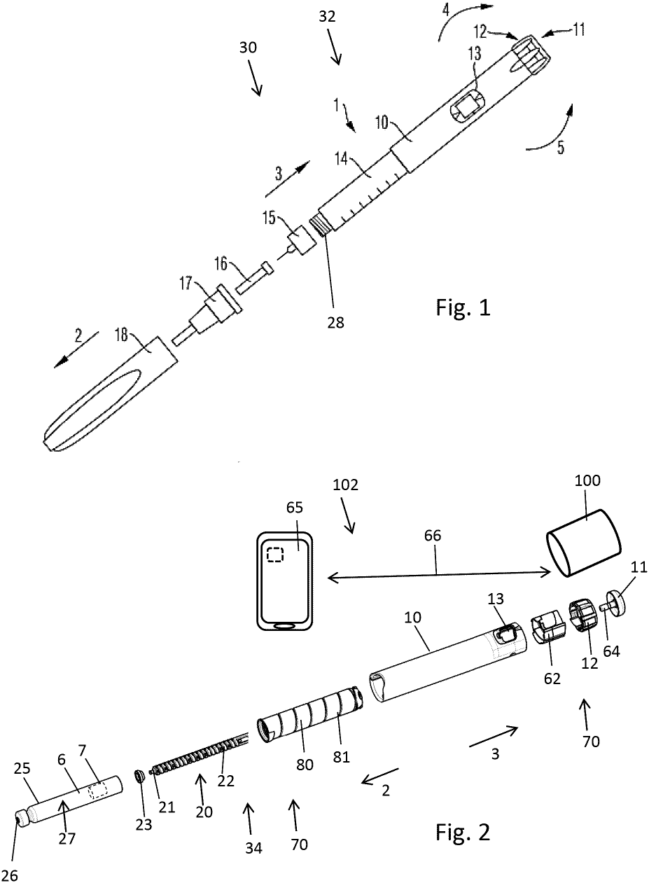

One example of a drug delivery device 1 for administering of a dose of a medicament 27 is illustrated in FIGS. 1 and 2. The drug delivery device 1 is implemented as an injection device 30. The injection device 30 is a handheld pen-type injector. The injection device 30 may be implemented as a disposable injection device 30. It may comprise a pre-filled cartridge 6 arranged inside a cartridge holder 14. With a disposable injection device 30, the cartridge holder 14 may be non-detachably connected to a body 10 of a housing 32 of the injection device 30.

With other examples, the injection device 30 is a re-usable injection device, wherein the cartridge holder 14 is detachably connected to the body 10 for replacing an empty cartridge 6. At or near a distal end of the housing 32, hence at the distal end of the cartridge holder 14, there is provided a socket 28 configured to mount or to engage with an injection needle 15. The socket 28 may be implemented as a threaded socket and the injection needle 15 may comprise a needle hub begin correspondingly threaded to provide a threaded engagement with the socket 28.

Typically, the injection needle 15 is protected by an inner needle cap 16 and either by an outer needle cap 17 and/or a protective cap 18 that is configured to enclose and to protect a distal section of the housing 32 of the injection device 30. The body 10 may comprise and form a main housing part configured to accommodate a drive mechanism 34 as shown in FIG. 2. The cartridge holder 14 may be regarded as a distal housing component of the injection device 30. The cartridge holder 14 may be permanently or releasably connected to the body 10 or main housing.

The cartridge 6 comprises a cylindrically-shaped or tubular-shaped barrel 25 sealed in proximal direction 3 by a bung 7 located inside the barrel 25. The cartridge six may be prefilled with a liquid medicament 27. The bung 7 is displaceable relative to the barrel 25 of the cartridge 6 in a distal direction 2 by means of a piston rod 20 of the drive mechanism 34. A distal end of the cartridge 6 is sealed by a pierceable seal 26 configured as a septum and being pierceable by a proximally directed tipped end of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU, wherein 1 IU is the biological equivalent of about 45.5 µg of pure crystalline insulin (1/22 mg). The dose dial 12 may comprise a sleeve shaped knob at the proximal end of the housing 32 of the injection device 30.

As shown further in FIGS. 1 and 2, the body 10 comprises a dosage window 13 that may be in the form of an aperture in the body 10. The dosage window 13 permits a user to view a limited portion of a number sleeve 80 that is configured to move when the dose dial 12 is turned. The number sleeve 80 and the dosage window 13 provide a visual indication of a dose currently set. The dose dial 12 may be rotated on a helical path with respect to the body 10 when turned during setting and/or dispensing or expelling of a dose.

With some other type of injection device, the dose dial 12 may be locked in longitudinal direction to the body 10. It may be then limited to a rotational movement relative to the body 10 for setting of the dose.

The injection device 30 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in the dosage window 13 will be ejected from injection device 1. When the needle 15 of the injection device 1 remains for a certain time in the skin portion after the trigger 11 is pushed, a high percentage of the dose is actually injected into the patient’s body. Ejection of an insulin dose may also cause a mechanical click sound, which is however different from the sounds produced when using the dose dial 12.

In the illustrated embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 30 may be used for several injection processes until either the cartridge 6 is empty or the expiration date of the medicament in the injection device 1 (e.g. 28 days after the first use) is reached.

At least some components of an example of a drive mechanism 34 are illustrated in more detail in FIG. 2. The drive mechanism 34 comprises numerous mechanically interacting components. A flange like support of the housing 10 comprises a threaded axial through opening threadedly engaged with a thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

Furthermore, there is provided a dose dial sleeve also denoted as number sleeve 80. The number sleeve 80 is located radially inward of the housing 10. A helical groove 81 is provided about an outer surface of the number sleeve 80. The body 10 is provided with the dosage window 13 through which a part of the outer surface of the number sleeve 80 can be seen. The body 10 is further provided with a helical rib at an inside sidewall portion of an insert piece 62, which helical rib is to be seated in the helical groove 81 of the number sleeve 80. The tubular shaped insert piece 62 is inserted into the proximal end of the tubular shaped body 10. Alternatively, such a helical rib may be also provided directly on an inside of the sidewall of the body 10. The helical rib as well as the insert piece 62 is rotationally and axially fixed to the body 10. There may be provided first and second stops on the body 10 to limit a dose setting procedure during which the number sleeve 80 is rotated in a helical motion relative to the housing 10.

The dose dial 12 in form of a dose dial grip is disposed about an outer surface of the proximal end of the number sleeve 80. An outer diameter of the dose dial 12 typically corresponds to and matches with the outer diameter of a proximal end of the body 10. The dose dial 12 is secured to the number sleeve 80 to prevent relative movement there between. The dose dial 12 is provided with a central opening.

A trigger 11, also denoted as dose button is substantially T-shaped. It is provided at a proximal end of the injection device 10. A stem 64 of the trigger 11 extends through the opening in the dose dial 12. The stem 64 and hence the trigger 11 is retained for limited axial movement relative to the number sleeve 80. A head of the trigger 11 is generally circular. The trigger side wall or skirt extends from a periphery of the head and is further adapted to be seated in a proximally accessible annular recess of the dose dial 12.

To dial a dose a user rotates the dose dial 12, along a dose incrementing direction 4, e.g clockwise. Dialing of a dose may be accompanied by a clicking sound. In this way, audible and/or tactile feedback of the dose being dialed is provided. Dialing of a dose is further accompanied by a rotation of the number sleeve 80, which starts to extend from the body 10 towards the proximal direction 3 when dialed along a dose incrementing direction 4, e.g in a clockwise sense.

The number sleeve 80, the dose dial 12 and the trigger may form part of a dial extension 70, hence and assembly of components of the drive mechanism 34 that starts to extend or to displace from the proximal end of the body 10 as a dose is dialed. During dispensing of a dose, hence when a user depresses the trigger 11 in distal direction 2, the dial extension 70 is subject to a distally directed movement relative to the body 10, hence along the distal direction 2. During such a dispensing motion, the number sleeve 80 is subject to a rotation along a dose decrementing direction 5, e.g. counter-clockwise.

The expelling mechanism or drive mechanism 34 as described above is only exemplary for one of a plurality of differently configured drive mechanisms that are generally implementable in a disposable or re-usable pen-injector. The drive mechanism as described above is explained in more detail e.g. in WO2004/078239A1, WO 2004/078240A1 or WO 2004/078241A1 the entirety of which being incorporated herein by reference.

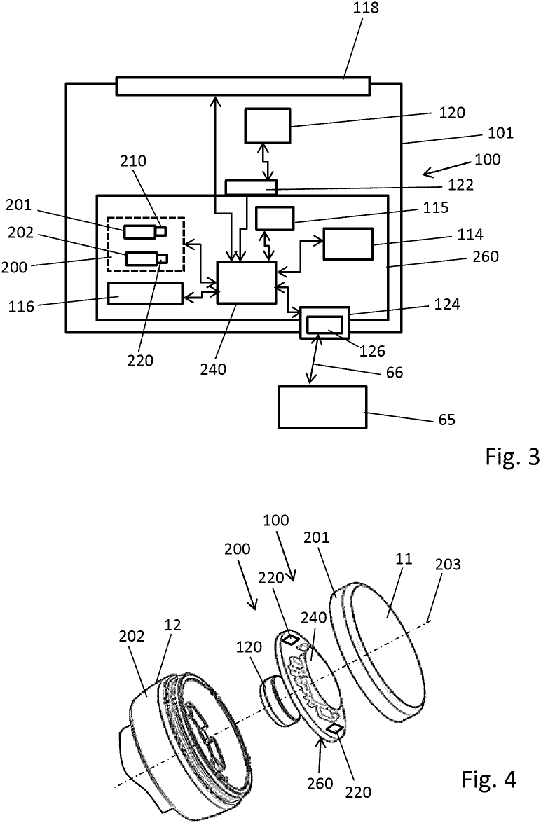

The drug delivery device 1, hence the injection device 30 may be further equipped with a data logging device 100 as indicated in FIGS. 3 or 4. The data logging device 100 may be integrated into the injection device 30. It may be positioned in a hollow receptacle provided by at least one of the trigger 11 and the dose dial 12. With other examples, the data logging device 100 is a separate component detachably connectable to the body 10 or to some other portion of the housing 32 of the injection device 30.

FIG. 2 is thus illustrative of a medicament injection system 102. The medicament injection system 102 comprises the injection device 30 and at least the data logging device 100. The data logging device 100 may be attachable to the injection device 30. It may be detachably connectable or detachably fixable to the housing 32 of the injection device 30. Alternatively, the data logging device 100 is integrated into the injection device 30. The medicament injection system 102 may further comprise the external electronic device 65. The external electronic device 65 may be implemented as a portable electronic device. It may comprise a smart watch, a smart phone, a tablet computer or a personal computer. The external electronic device 65 and the data logging device 100 are configured to establish a communication link 66, e.g. a wireless communication link, in order to transfer data therebetween.

The data logging device 100 as schematically indicated in FIG. 3 typically comprises a sensor, e.g. in form of rotation sensing arrangement 200, by way of which a relative rotation of at least one component of the drug delivery device 1 relative to another component of the drug delivery device 1 can be determined or quantitatively measured, e.g. during at least one of setting of the dose and dispensing of the dose.

The data logging device 100 may be either integrated into the drug delivery device 1 or may be provided as a separate add-on device. When provided as a separate add-on device the data logging device 100 may be provided with an own housing 101 attachable to the housing 32 of the drug delivery device 1 or injection device 30.

The data logging device 100 comprises one or more processors 240, such as a microprocessor, a Digital Signal Processor (DSP), Application Specific Integrated Circuit (ASIC), Field Programmable Gate Array (FPGA) or the like, together with a memory 114. The memory may 114 include a program memory and main memory, which can store software for execution by the processor 240 and data generated or captured during use of the data logging device 100 such as counted pulses, derived dose size, time stamp, etc. An optional switch 122 connects a power source 120 to the electronic components of the data logging device 100, including the rotation sensing arrangement 200. A display 118 may or may not be present. The rotation sensing arrangement 200 may be coupled to a first member 201 and a second member 202. It comprises at least one sensor 220 connected or attached to the second member 202 and further comprises at least one signal generator 210 connected or attached to the first member 201.

With the present implementation of the rotation sensing arrangement 200 in a data logging device 100 one of the first member 201 and the second member 202 can be connected or fastened to the housing 32 of the injection device 30 and the other one of the first member 201 and the second member 202 is for instance connectable or fastenable to the rotatable dose dial 12 of the injection device 30.

The data logging device 100 typically comprises an interface 124 connected to the processor 240. The interface 124 may be a wireless communications interface for communicating with another external electronic device 65, e.g. in form of a portable electronic device, via a wireless communication protocol or network such as Wi-Fi or Bluetooth®, RFID or NFC (near field communication). The wireless communication interface may be operable in the radio frequency range. For instance, the wireless communication interface may be based on radio-frequency identification technology (known as RFID) which allows compatible hardware to both supply power to and communicate with an otherwise unpowered and passive electronic tag using radio waves. It may be hence used for identification, authentication and tracking.

With other examples, the interface 124 is implemented as a wired communications link, such as a socket for receiving a Universal Series Bus (USB), mini-USB or micro-USB connector. For this, the interface 124 comprises a transceiver 126 configured for transmitting and receiving data. FIG. 3 depicts an example of an injection system in which the add-on device 100 is connected to an external electronic device 65, via a communication link 66 for data transfer. The data connection 66 may be of wired or wireless type.

For example, the processor 240 may store determined delivered medicament amounts and time stamps for the injections as they are administered by the user and subsequently, transfer that stored data to the external electronic device 65. The device 65 maintains a treatment log and/or forwards treatment history information to a remote location, for instance, for review by a medical professional.

The add-on device 100 or data collection device may be configured to store data such as delivered medicament amounts and time stamps of numerous injection events, such as 35 or more injection events. According to a once-daily injection therapy this would be sufficient to store a treatment history of about one month. The data memory 114 may be organized in a first-in first-out manner ensuring that most recent injection events are always present in the memory of the data collection device 100. Once transferred to an external electronic device 65 the injection event history in the add-on device 100 might be deleted. Alternatively, the data remains in the add-on device 100 and the oldest data is deleted automatically once new data is stored. This way the log in the data collection device is built up over time during usage and will always comprise the most recent injection events. Alternatively, other configuration could comprise a storage capacity of 70 (twice daily), 100 (three months) or any other suitable number of injection events depending on the therapy requirements and/or the preferences of the user.

In another embodiment, the interface 124 may be configured to transmit information using a wireless communications link and/or the processor 240 may be configured to transmit such information to the external electronic device 65 periodically.

The processor 240 may control the optional display 118 to show the determined medicament dose information, and/or to show an elapsed time since a last medicament dose was delivered. For example, the processor 240 may cause the display 118 to switch periodically between displaying the most recent determined medicament dosage information and the elapsed time.

The power source 120 may be a battery. The power source 120 may be a coin cell, or multiple coin cells arranged in series or parallel. A timer 115 may be also provided. In addition to, or instead of, switching the add-on device 100 on and off, the switch 122 may be arranged to trigger the timer 115 when engaged and/or disengaged. For example, if the timer 115 is triggered on both engagement or disengagement of the first and second electrical contacts of the switch or both operation and ceasing of operation of the switch 122, then the processor 240 may use the output from the timer 115 to determine a length of time during which the trigger 11 was pressed, for example to determine the duration of an injection.

Alternatively, or additionally, the processor 240 may use the timer 115 to monitor a length of time that has elapsed since an injection was completed, as indicated by a time of disengagement of respective switch components or ceasing of operation of the switch 122. Optionally, the elapsed time may be shown on the display 118. Also optionally, when the switch 122 is next operated, the processor 240 may compare the elapsed time with a predetermined threshold, to determine whether a user may be attempting to administer another injection too soon after a previous injection and, if so, generate an alert such as an audible signal and/or a warning message on the display 118 or via the output 116. The output 160 may be configured to generate an audible sound or to induce a vibration hence to produce a tactile signal, e.g. for alerting the user.

The sensing arrangement 200 as shown in FIG. 3 might be implemented as a rotation sensing arrangement. It may comprise a first member 201 and a second member 202. The first member 201 is rotatable relative to the second member 202 with regards to an axis of rotation 203. Typically, the first member 201 and the second member 202 are arranged coaxial with regards to the axis of rotation 203. With some examples, the first member 201 and the second member 202 are arranged axially adjacent with regard to the axis of rotation 203. The first member 201 and the second member 202 may be directly mechanically engaged. With other examples, the first member 201 and the second member 202 are mechanically disengaged from each other. Here, the first member 201 and the second member 202 may be separately arranged or rotationally supported in or at a housing 32 of the injection device 30 or in or at a respective housing 101 of a separate add-on device 100.

At least one of the first member 201 and the second member 202 is typically rotationally supported in or on the housing 10 of the injection device 1. With some examples, both, the first member 201 and the second member 202 can be rotationally supported on or with regard to the housing 32. Typically, and depending on the specific implementation or integration of the rotation sensing arrangement 200 in the injection device 30 one of the first member 201 and the second member 202 is rotationally locked to the housing 32 whereas the other one of the first member 201 and the second member 202 is rotationally movable relative to the housing 10. Typically, one of the first member 201 and the second member 202 is rotatable relative to the housing 32 with regard to the axis of rotation 203. The second member 202 may be represented by the dose dial 12. The first member 201 may be represented by the trigger 11. During at least one of setting and dispensing of the dose, the second member 202 is subject to a rotation relative to the first member 201. A degree of relative rotation between the first member 201 and the second member 202 is directly indicative of the size of the dose actually set and/or dispensed.

The rotation sensing arrangement 200 may comprise a printed circuit board 260 as illustrated in FIG. 4. On the printed circuit board 260 there may be provided the processor 240 together with the at least one sensor 220. The printed circuit board 260 may be further provided with a power source 120. The power source 120 may be located on one side of the printed circuit board 260. On the same side or on the opposite side of the printed circuit board 260 there may be provided the processor 240 and/or the at least one sensor 220.

In order to provide sufficient data security establishing of a communication link 66 between the data logging device 100 and an external electronic device 65 requires use of a pairing code 320. Due to regulatory requirements the pairing code 320 may have to be visible or discernible from outside the drug delivery device 1. However, it should not be directly visible to any person gaining access to the drug delivery device 1. In order to prevent unauthorized pairing of the electronic data logging device 100 with the external electronic device 65 the pairing code 320 is either concealed by a component of the drug delivery device 1 and has to be revealed by an action of a user. According to other options, the pairing code 320 is split or distributed on at least two device components 301, 302. For recombining of the pairing code 320, the first and second device components 301, 302 will have to be moved into a predefined positional state.

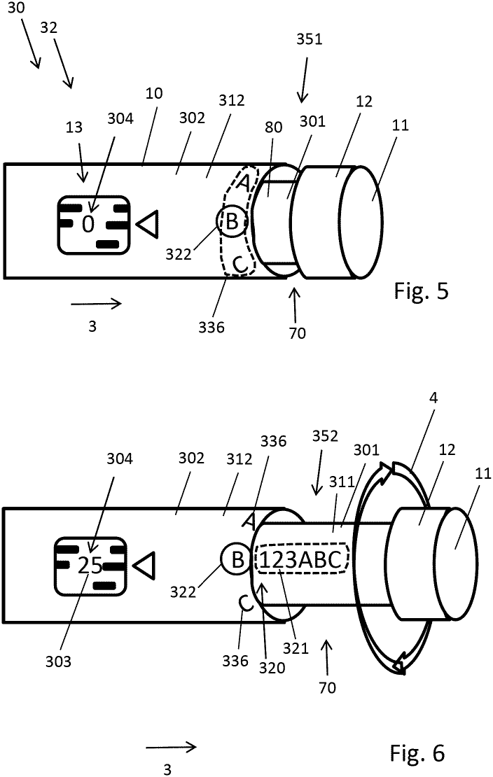

In the example of FIGS. 5 and 6, at least one of a dial extension 70 and a number sleeve 80 provides a first device component 301 movable relative to a second device component 302. The second device component 302 may be provided by the body 10 of the housing 32 of the injection device 30. The first device component 301 is at least partially contained or received inside a hollow section of the second device component 302. As it is apparent from a comparison of FIGS. 5 and 6, the first device component 301 is displaceable or movable relative to the second device component 302 along a longitudinal direction, hence towards a proximal direction 3. Optionally, the first device component 301 may be also rotatable relative to the second device component 302.

Mutual engagement of the first and second device components 301, 302 may only allow and support a helical motion of the first device component 301 relative to the second device component 302. The first device component 301 is provided with a first outer surface 311. On the first outer surface 311 there is provided at least a first code portion again 321 of a pairing code 320. Typically, the entirety of the pairing code is required for establishing or for setting up of the communication link 66 between the data logging device 100 and the external electronic device 65.

The second device component 302 comprises a second outer surface 312. On the second outer surface 312 there is provided a second code portion 322. A recombination of the second code portion 322, hence the character “B” and the sequence of numbers and characters “123ABC” of the first code portion 321 complement or constitute the pairing code 320. The pairing code 320 is only complemented and recombined from the first code portion 321 and the second code portion 322 when the first device component 301 is in a predefined second positional state 352 relative to the second device component 302.

This particular positional state is characterized by a relative position of first and second device component 301, 302 and/or by a predefined rotational orientation of the first and second device components 301, 302 relative to each other. The second positional state 352 may be characterized and may be identified by a first marker 303 of the first device component 303 configured to coincide, to align or to spatially overlapping with a second marker 304 provided by the second device component 302. Here, the second marker 304 is an aperture in the sidewall of the first device component 302. The aperture 304 may coincide with the above-mentioned the dosage window 13. The first marker 303 may be a selected or predefined number or symbol of numerous available numbers or symbols as provided on the outer surface 311 of the first device component 301, e.g. implemented as a number sleeve 80. In the presently illustrated example, the first marker 303 is number 25, also indicating that a dose of 25 units has been actually set in the present configuration of first and second device components 301, 302.

In the presently illustrated example also the second device component 302 is provided with a second code portion 322. The second code portion 322 and the first code portion 321 have to be aligned along a straight line or along a helical line so as to reconstitute the pairing code 320 required for establishing or for setting up the communication link 66.

With the presently illustrated example, the second code portion 322 is provided on the second outer surface 312 in combination with a second concealing code portion 336. The second concealing code portion 336 provides some further or supplemental code portion that is generally indistinguishable from the second code portion 322. The second concealing code portion 336 may comprise numerous characters, symbols or numbers aligned with the second code portion 322. When not being appropriately informed, e.g. by a user manual of the drug delivery device, it is not known to authorized user of the drug delivery device 1, which one of the second concealing code portions 336, e.g. letters “A” or “C” and the second code portion 322, character “B” should align with the first code portion 321 in order to recombine and to constitute the pairing code 320.

In the example of FIGS. 5 and 6, the second concealing code portion 336 extends along a second direction 4. The second direction may extend at a predefined angle relative to a first direction 3. The first code portion extends along the first direction 3 and the second concealing code portion 336 extends along or parallel to the second direction 4. When the first device component 301 is subject to both, a movement along the first direction 3 and along the second direction 4 relative to the second device component 302 for arriving at the second positional state 352 an intersection of a longitudinal extension of the first code portion 321 with the second concealing code portion 336 may point to or indicate the second code portion 322.

In the presently illustrated example and when for instance a dose of the medicament should be dialed that does not match with the first marker 303, there will be an alignment mismatch between the first code portion 321 and the second code portion 322 and thus the pairing code 320 will not show up appropriately.

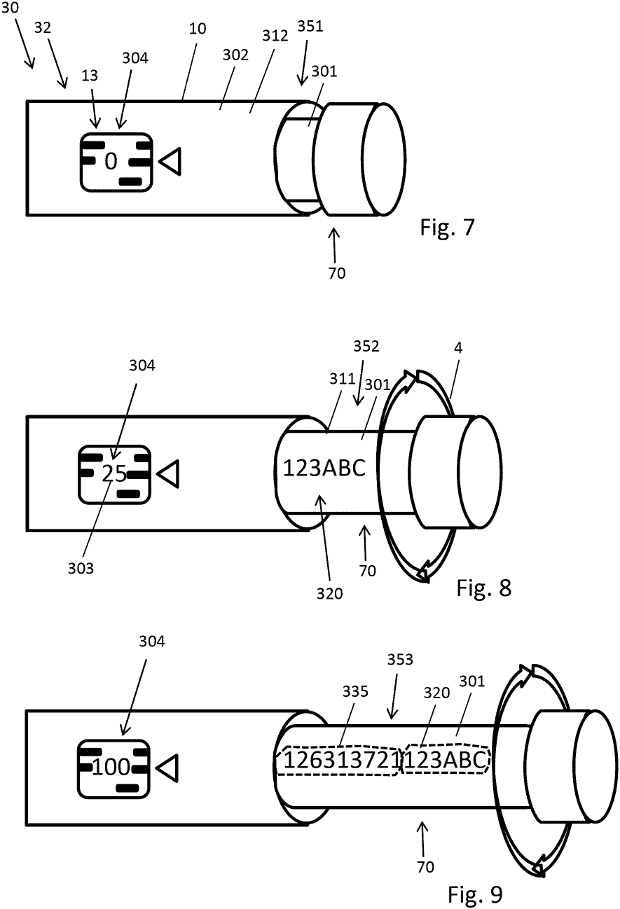

In another example as illustrated in the sequence of FIGS. 7-9, the second device component 302 is void of a second code portion 322 or a second concealing code portion 336. There, the second device component 302 is opaque and receives at least a portion of the first device component 301 in its hollow interior. In the first positional state 351 as illustrated in FIG. 7, the first outer surface 311 of the first device component 301 is hardly visible from outside the injection device 30. It is upon a movement of the first device component 301 relative to the second device component 302, that a portion of the first outer surface 311 is revealed from the second device component 302.

The movement of the first device component 301 relative to the second device component 302 from the first positional state 351 as shown in FIG. 7 towards and into the second positional state 352 as shown in FIG. 8 may be conducted in the course of setting of a dose of the medicament. Hence, the user manual for pairing of the electronic data logging device 100 and the external electronic device 65 may instruct the user of the injection device 30 to dial a number of 25 standard units. This dialing or dose setting motion, e.g. conducted by the movement of the first device component 301 relative to the second device component 302, leads to a combined rotational and longitudinal displacement of the first device component 301 relative to the second device component 302.

As illustrated in FIG. 8, the first device component 301 protrudes in longitudinal direction from a longitudinal end, hence from the proximal end of the first device component 302. As a consequence, a respective portion of the first outer surface 311 appears. When arriving at the second positional state 352 as illustrated in FIG. 8, it is only the entirety of the pairing code 320 that is visually identifiable from outside the drug delivery device 30. Here, it is only and exclusively the pairing code 320 that is revealed.

As it is apparent from the further illustration of FIG. 9, and hence when the first device component 301 is moved further and beyond the second positional state, e.g. along the proximal direction 3 relative to the first device component 302 a further portion of the first outer surface 311 reveals. The further portion of the first outer surface 311 is provided with a first concealing code portion 335. The first concealing code portion 335 is indistinguishable from the first code portion 321 or from the pairing code 320. Hence, in the configuration as shown in FIG. 9, a user of the device will be unable to visually identify which part of the indistinguishable combination of the pairing code 320 and the first concealing code portion 335 should be used or which part thereof is relevant for initiating or approving the pairing between the data logging device 100 and the external electronic device 65.

With typical implementations of the drug delivery device 30, the first device component 301 is movable relative to the second device component 302 from the first positional state 351, such as illustrated in FIGS. 5 and 7 to the second positional state 352 as illustrated in FIGS. 6 and 8 and vice versa from the second positional state 352 towards and into the first positional state 351 without the necessity to dispense a dose of the medicament 27.

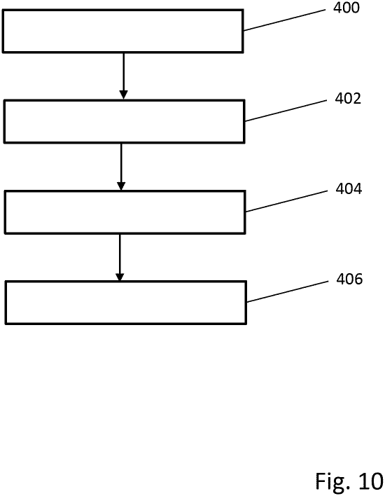

In FIG. 10, a flowchart of a method of pairing of the data logging device 100 with the external electronic device 65 is illustrated. Typically, the method of pairing can be conducted with a drug delivery device 1 or with an injection device 30 as described above in connection with an external electronic device 65 and a data logging device 100, either provided as a separate data logging device 100 or integrated into the drug delivery device 1.

For setting up a communication link 66 between the data logging device 100 and the external electronic device 65 in a first step 400, at least one of the external electronic device 65 and the data logging device 100 is set into a pairing mode. Typically, the external electronic device 65 and the data logging device 100 are configured to set up a wireless communication link, e.g. using a standardized RF connection protocol, such as Bluetooth. In a subsequent step 402 the first device component 301 is moved relative to the second device component 302 from a first positional state 351, in which the pairing code 320 is non-discernible, into the second positional state 352. In a subsequent step 404, the pairing code 320 is visually identified by a user of the drug delivery device. In a further step 406, the pairing code 320 as identified by the user in step 404 is used to establish or to approve the communication link 66 between the external electronic device 65 and the data logging device 100.

List of reference numbers
1 drug delivery device
2 distal direction
3 first direction
4 second direction
5 dose decrementing direction
6 cartridge
7 bung
8 drive mechanism
9 dose setting mechanism
10 body
11 trigger
12 dose dial
13 dosage window
14 cartridge holder
15 injection needle
16 inner needle cap
17 outer needle cap
18 protective cap
19 protrusion
20 piston rod
21 bearing
22 threaded section
23 pressure foot
25 barrel
26 seal
27 medicament
28 socket
30 injection device
32 housing
34 drive mechanism
62 insert piece
64 stem
65 external electronic device
66 communication link
70 dial extension
80 number sleeve
81 groove
100 data logging device
101 housing
102 medicament injection system
114 memory
115 timer
116 output
118 display
120 power source
122 switch
124 interface
126 transceiver
200 sensing arrangement
201 first member
202 second member
203 axis of rotation
210 signal generator
220 sensor
240 processor
260 printed circuit board
301 device component
302 device component
303 marker
304 marker
311 outer surface
312 outer surface
320 pairing code
321 code portion
322 code portion
335 code portion
336 code portion
351 positional state
352 positional state
353 positional state

1-15. (canceled)

16. A drug delivery device for administering of a dose of a medicament, the drug delivery device being operable to cooperate with an electronic data logging device, the drug delivery device comprising:
a housing configured to accommodate the medicament and configured to receive and/or mechanically engage with the electronic data logging device; and
a first device component and a second device component,
wherein the first device component is movable relative to the second device component from a first positional state to a second positional state,
wherein the first device component comprises a first outer surface,
wherein the second device component comprises a second outer surface,
wherein at least one of the first outer surface and the second outer surface comprises a first code portion of a pairing code for establishing a communication link between the electronic data logging device and an external electronic device, and
wherein the pairing code is visually identifiable from outside the drug delivery device only when the first device component is in the second positional state relative to the second device component.

17. The drug delivery device according to claim 16, wherein the pairing code is non-discernible from outside of the drug delivery device when the first device component is in the first positional state.

18. The drug delivery device according to claim 16, wherein the first code portion or the pairing code is provided on the first outer surface, and wherein at least a part of the first code portion or the pairing code is concealed by the second device component when the first device component is outside the second positional state .

19. The drug delivery device according to claim 16, wherein the first outer surface is provided with one of the first code portion and the pairing code, wherein the first outer surface is further provided with a first concealing code portion that is indistinguishable from the first code portion or from the pairing code, wherein an indistinguishable composition of at least a portion of the first concealing code portion and at least one of the first code portion and the pairing code is discernible from outside the drug delivery device when the first device component is outside the second positional state.

20. The drug delivery device according to claim 19, wherein the first concealing code portion is concealed by the second device component when the first device component is in the second positional state.

21. The drug delivery device according to claim 16, wherein the pairing code comprises the first code portion and a second code portion, wherein the first code portion is provided on the first outer surface, and wherein the second code portion is provided on the second outer surface.

22. The drug delivery device according to claim 21, wherein the first code portion and the second code portion mutually recombine and constitute the pairing code when the first device component is in the second positional state.

23. The drug delivery device according to claim 16, wherein at least one of the pairing code and the first code portion extends along a first direction, and wherein the first device component is movable along the first direction relative to the second device component.

24. The drug delivery device according to claim 21, wherein the second outer surface is further provided with a second concealing code portion that is indistinguishable from the second code portion, and wherein the first code portion aligns with the second code portion when the first device component is in the second positional state.

25. The drug delivery device according to claim 24, wherein the first code portion or a fraction thereof aligns with the second concealing code portion when the first device component is outside the second positional state.

26. The drug delivery device according to claim 24, wherein at least one of the second concealing code portion and the second code portion extends along a second direction, and wherein the first device component is movable along the second direction relative to the second device component.

27. The drug delivery device according to claim 16, further comprising a first marker and a second marker, wherein the first marker is provided by or is connected to the first device component, wherein the second marker is provided by or is connected to the second device component, wherein the first marker is subject to a movement relative to the second marker when the first device component moves relative to the second device component, and wherein the first marker and the second marker visually align, visually overlap, or mechanically engage when the first device component reaches the second positional state relative to the second device component.

28. The drug delivery device according to claim 16, wherein the first device component is one of a dose dial, a dial extension, a number sleeve, and a protective cap of a pen-type injection device.

29. The drug delivery device according to claim 16, wherein the second device component is one of a housing, a body of the housing, and a cartridge holder of a pen-type injection device.

30. The drug delivery device according to claim 16, further comprising a cartridge filled with a medicament and arranged inside the housing.

31. A method of pairing an electronic data logging device of a drug delivery device with an external electronic device, wherein the drug delivery device comprises a first device component movable relative to a second device component, wherein the first device component comprises a first outer surface, wherein the second device component comprises a second outer surface, wherein at least one of the first outer surface and the second outer surface comprises a first code portion of a pairing code, the pairing code being visually identifiable from outside the drug delivery device only when the first device component is in a second positional state relative to the second device component, and wherein the method comprises:
setting at least one of the external electronic device and the data logging device into a pairing mode;
moving the first device component relative to the second device component from a first positional state, in which the pairing code is non-discernible, into the second positional state;
visually identifying the pairing code from outside the drug delivery device; and
using the pairing code to establish a communication link between the external electronic device and the data logging device.

32. The method according to claim 31, wherein the pairing code is non-discernible from outside of the drug delivery device when the first device component is in the first positional state.

33. The method according to claim 31, wherein the first code portion or the pairing code is provided on the first outer surface, and wherein at least a part of the first code portion or the pairing code is concealed by the second device component when the first device component is outside the second positional state.

34. The method according to claim 31, wherein the first outer surface is provided with one of the first code portion and the pairing code, wherein the first outer surface is further provided with a first concealing code portion that is indistinguishable from the first code portion or from the pairing code, wherein an indistinguishable composition of at least a portion of the first concealing code portion and at least one of the first code portion and the pairing code is discernible from outside the drug delivery device when the first device component is outside the second positional state.

35. The method according to claim 31, wherein the pairing code comprises the first code portion and a second code portion, wherein the first code portion is provided on the first outer surface, and wherein the second code portion is provided on the second outer surface.
